(19) United States
(12) Patent Application Publication
(10) Pub. No.: US 2021/0145316 A1
(43) Pub. Date: May 20, 2021

(54) DENTAL CALIPER AND METHOD

(71) Applicant: Groman Inc., Margate, FL (US)

(72) Inventor: Boaz Barry Groman, Boca Raton, FL (US)

(21) Appl. No.: 17/098,445

(22) Filed: Nov. 15, 2020

Related U.S. Application Data
(60) Provisional application No. 62/935,649, filed on Nov. 15, 2019.

(51) Int. Cl. A61B 5/107; A61C 19/05

(52) U.S. Cl. CPC A61B 5/1072; A61C 19/05

(57) ABSTRACT

Dental caliper and method for determining occlusion. The caliper may comprise an elongate member, or “beam”, marked with a scale (indicia); a first jaw (or probe) extending from the beam, and fixed to the beam; and a second jaw (or probe) extending from the beam, and movable along the beam; wherein: a proximal portion of the first (fixed) jaw is offset from a distal portion of the jaw. The offset may be is fixed, such as 3 mm, or may have a gradient, such as 3-5 mm. The method may comprise using the caliper to (i) measure the eye-to-ear distance and (ii) measure the nose-to-chin distance. This may be done without adjusting the probes. The caliper and method of use may represent an improvement over U.S. Pat. No. 4,718,850 (Knebelman).

### CROSS REFERENCE TO RELATED APPLICATIONS

Priority (filing date benefit) is claimed, from the following:

nonprovisional of 62935649 filed 15 Nov. 2019

### FIELD OF THE INVENTION

The invention relates to measuring devices, more particularly to a caliper-type tool for measuring distanced between features on a patient's face.

### BACKGROUND

A caliper is a device (or tool, or instrument) used to measure the distance between two points, such as opposite sides of an object. This may be the outside diameter (OD) of a pipe, the inside diameter (ID) or a cylinder, or the distance between any two points of interest.

Many types of calipers permit reading out a measurement on a ruled scale, a dial, or a digital display. A caliper may be referred to as a gauging device.

A caliper may have two jaws (or probes, or arms) extending from an elongate member. Typically one of the jaws is fixed to the elongate member, the other jaw is movable along the elongate member. A scale with indicia, such as a millimeter scale may be provided on the elongate member so that the distance between the jaws can be read. (Some calipers have digital readout devices incorporated therein rather than, or in addition to the indicia on the elongate member.)

A conventional caliper may have an elongate member with two sets of probes. One of each set of probes may be fixed to the elongate member, the other may be movable back and forth on the elongate member. A scale may be provided on the elongate member for displaying a measurement. The caliper may include:

a first set of probes may extend in one direction (such as downward) from the elongate member and may be suitable for being positioned on the outside of an object, such as for measuring the outside diameter (OD) of the object, or the distance between two exterior points on the object. The maximum size object that may be measured in this manner may be limited by the length of the elongate member.

a second set of probes may extend in another direction (such as upward) from the elongate member and may be suitable for being positioned on the inside of an object, such as for measuring the inside diameter (OD) of the object, or the distance between two interior points on the object. The minimum size object that may be measured in this manner may be limited by the size of the probes, which must fit within the space between the two points being measured on the object.

Typically, in use, the caliper is positioned so that the two jaws (or probe tips, or the like) are disposed at the two points (i.e., the distance therebetween) being measured, the caliper is removed and the distance is read. Of course, the distance may be read with the caliper yet engaged with the object being measured.

U.S. Pat. No. 4,718,850 (Knebelman, Jan. 12, 1988), incorporated by reference herein, discloses a type of caliper and method. Vertical dimension of occlusion, i.e. the vertical dimension of the face with the posterior teeth fitting tightly together, is readily determined by a method comprising first measuring the distance between the external auditory meatus and the lateral corner of the ocular orbit, making an adjustment in the first measurement to arrive at the factored distance and then positioning the mandible so that the distance between the nasal spine and the anterior part of the undersurface of the mandible corresponds to the factored distance. Gauging devices are disclosed for performing the method. More particularly, as set forth in the claims (annotated with “step #” herein).

A method for determining vertical dimension of occlusion comprising the steps of:

providing a gauging device comprising a first probe terminating in a first tip portion affording registry of said first tip with selected anatomical parts, and a second probe adjustably mounted substantially parallel to said first probe for adjustment relative to said first probe and said second probe having a second tip portion affording registry of said second tip with selected anatomical parts;

step 1) first registering one of said tips with the external auditory meatus, and adjusting said gauging device to register the other of said tips with the lateral corner of the ocular orbit to establish a first measurement;

step 2) then positioning the tip of the second probe spaced relative to the tip of said first probe a factored distance based on said first measurement;

step 3) thereafter, without changing said positioning of the tips, registering one of said tips with the nasal spine and positioning the other of said tips adjacent the anterior part of the undersurface of the mandible; and step 4) then positioning the mandible to engage said other tip.

In other words (plain English), in Knebelman, the dentist performs the following sequence of steps with a caliper Step 1. measure the eye-to-ear distance Step 2. adjust the probes based on “Table A” to establish an offset Step 3. measuring the nose-to-chin distance Step 4. positioning the mandible to engage said other tip

See also http://craniometers.com/ for a presentation of how Knebelman works. See APPENDIX (aka FIG. 4)

### SUMMARY

It is an object of the invention, generally, to provide an improved caliper for measuring the distance between features on a patient's face.

It is an object of the invention to provide a purpose-specific caliper and an improved method for performing measurements, such as the dimension of occlusion.

According to the invention, generally, a dental caliper and method for determining occlusion is disclosed. The caliper may comprise an elongate member, or “beam”, marked with a scale (indicia); a first jaw (or probe) extending from the beam, and fixed to the beam; and a second jaw (or probe) extending from the beam, and movable along the beam; wherein: a proximal portion of the first (fixed) jaw is offset from a distal portion of the jaw. The offset may be is fixed, such as 3 mm, or may have a gradient, such as 3-5 mm. The method may comprise using the caliper to (i) measure the eye-to-ear distance and (ii) measure the nose-to-chin distance. This may be done without adjusting the probes. The caliper and method of use may represent an improvement over U.S. Pat. No. 4,718,850 (Knebelman).

According to some embodiments (examples) of the invention, a dental caliper for determining occlusion may comprise: an elongate member, or “beam”, marked with a scale (indicia); a first jaw (or probe) extending from the beam, and fixed to the beam; and a second jaw (or probe) extending from the beam, and movable along the beam; wherein: a proximal portion of the first (fixed) jaw is offset from a distal portion of the jaw. The offset may be fixed, such as 3 mm. The offset may have a gradient, such as 3-5 mm.

According to some embodiments (examples) of the invention, a method of determining vertical dimension of occlusion may comprise: using the caliper of claim 1, performing the following steps: Step 1. measuring the eye-to-ear distance (corresponds with Knebelman Step 1); and Step 2. measuring the nose-to-chin distance (corresponds with Knebelman Step 3); and this may be done without adjusting the probes (compare Knebelman Step 2).

Other objects, features and advantages of the invention may become apparent in light of the following description(s) thereof.

### BRIEF DESCRIPTION OF THE DRAWINGS

Reference will be made in detail to embodiments of the disclosure, non-limiting examples of which may be illustrated in the accompanying drawing figures (FIGS). The figures may generally be in the form of diagrams. Some elements in the figures may be stylized, simplified or exaggerated, others may be omitted, for illustrative clarity.

Although the invention is generally described in the context of various exemplary embodiments, it should be understood that it is not intended to limit the invention to these particular embodiments, and individual features of various embodiments may be combined with one another. Any text (legends, notes, reference numerals and the like) appearing on the drawings are incorporated by reference herein.

FIG. 1 is a diagram (plan view) of a caliper, according to the invention.

FIG. 2A is a diagram (plan view) of a portion of a caliper, with a “fixed” correction feature, according to the invention.

FIG. 2B is a diagram (plan view) of a portion of a caliper, with a “gradient” correction feature, according to the invention.

FIG. 3 is a diagram (perspective view) of a caliper, according to the invention.

### DETAILED DESCRIPTION

Various embodiments (or examples) may be described to illustrate teachings of the invention(s), and should be construed as illustrative rather than limiting. It should be understood that it is not intended to limit the invention(s) to these particular embodiments. It should be understood that some individual features of various embodiments may be combined in different ways than shown, with one another. Reference herein to “one embodiment”, “an embodiment”, or similar formulations, may mean that a particular feature, structure, operation, or characteristic described in connection with the embodiment is included in at least one embodiment of the present invention. Some embodiments may not be explicitly designated as such (“an embodiment”).

The embodiments and aspects thereof may be described and illustrated in conjunction with systems, devices and methods which are meant to be exemplary and illustrative, not limiting in scope. Specific configurations and details may be set forth in order to provide an understanding of the invention(s). However, it should be apparent to one skilled in the art that the invention(s) may be practiced without some of the specific details being presented herein. Furthermore, some well-known steps or components may be described only generally, or even omitted, for the sake of illustrative clarity. Elements referred to in the singular (e.g., “a widget”) may be interpreted to include the possibility of plural instances of the element (e.g., “at least one widget”), unless explicitly otherwise stated (e.g., “one and only one widget”).

In the following descriptions, some specific details may be set forth in order to provide an understanding of the invention(s) disclosed herein. It should be apparent to those skilled in the art that these invention(s) may be practiced without these specific details. Any dimensions and materials or processes set forth herein should be considered to be approximate and exemplary, unless otherwise indicated. Headings (typically underlined) may be provided as an aid to the reader, and should not be construed as limiting.

It is an object of the invention to provide a purpose-specific caliper and an improved method for performing measurements, such as the dimension of occlusion.

The method disclosed herein eliminates Step 2 of Knebelman, thereby eliminating the need to adjust the probes (Step 2) after making the first measurement (Step 1. eye-to-ear).

The method may be facilitated by a caliper with a built-in offset, essentially replacing (eliminating) Step 2 (adjust the probes).

According to an aspect of the invention, generally, a caliper particularly suited to measuring the dimension of occlusion comprises:

an elongate member with a scale

a first jaw (or probe) extending from the elongate member (or beam)

a second jaw (or probe) extending from the elongate member (or beam)

The first jaw may be fixed to the elongate member (or beam), and may have a special profile with a built-in offset, such as may be discussed in greater detail hereinbelow.

The second jaw may be movable along the elongate member (or beam), and may be provided with means (such as an arrow, a dot, a line, or the like, or simply a surface of the second jaw overlapping the scale on the elongate member) for indicating (in cooperation with the scale on the elongate member) the distance between the two jaws—i.e., the distance being measured.

According to an aspect of the invention, generally, a method for determining vertical dimension of occlusion may comprise the steps of:

Step 1. measure the eye-to-ear distance (corresponds with Knebelman Step 1)

Step 2. measuring the nose-to-chin distance (corresponds with Knebelman Step 3)

Note that Steps 2 and 4 of Knebelman have been eliminated.

Note that “determining” a parameter of interest (such as vertical occlusion), as used herein, may involve measuring different (other) parameters (such as eye-to-ear distance and nose-to-chin distance) that are correlated with the parameter of interest—in other words, in a sense, indirectly measuring the parameter of interest.

### The Caliper

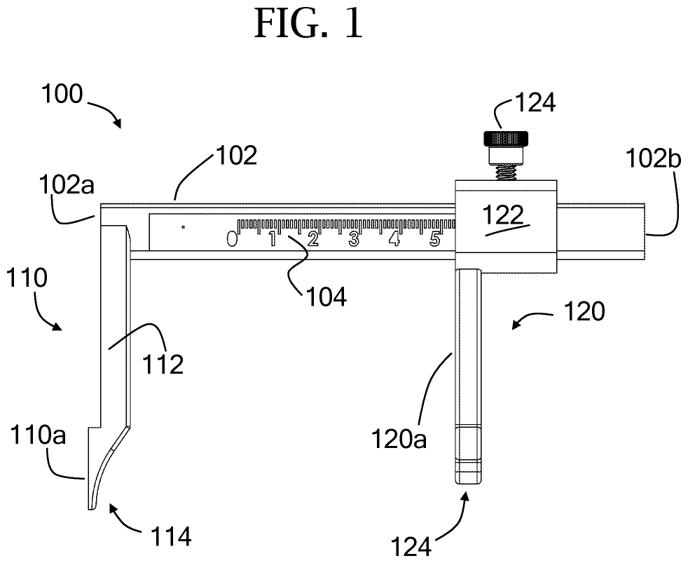

FIG. 1 shows a dental caliper, according to an embodiment of the invention.

The caliper 100 comprises an elongate member 102 having two ends 102a/102b, and marked with a scale 104, such as a millimeter scale. The elongate member may be referred to as a “rod”, or “beam”, or the like. The elongate member has a longitudinal axis.

A first jaw (or probe) 110 extends from an end (left, as viewed, 102a) of the elongate member 102 and is fixed (not movable) with respect to the elongate member.

A second jaw (or probe 120) extends from the elongate member 102 and is movable along the elongate member 102, generally from one end to the other. It is shown extending from a mid-portion of the elongate member.

The second (movable) jaw 120 has a simple straight profile, having a surface 120a (left, as viewed) which is substantially straight, and may extend substantially perpendicular to the elongate member 102. This left, or inner surface 120a of the second jaw, or the tip 124 thereof, may be disposed at or upon a first point being measured.

The first (fixed) jaw 110 has a special profile, having a surface 110a (left, as viewed) which is not straight, and may extend generally perpendicular to the elongate member 102. This left, or outer surface 110a of the first jaw, or the tip 113 thereof, may be disposed at or upon a second point being measured

More particularly, the first jaw 110 may comprise a main body portion 112 extending from the left end 102a of the elongate member 102 and a tip portion 114. The main body portion may be integrally formed with the elongate member so that it is fixed thereto, and not movable. The tip portion 114 may be disposed at a distal end of the body portion 112.

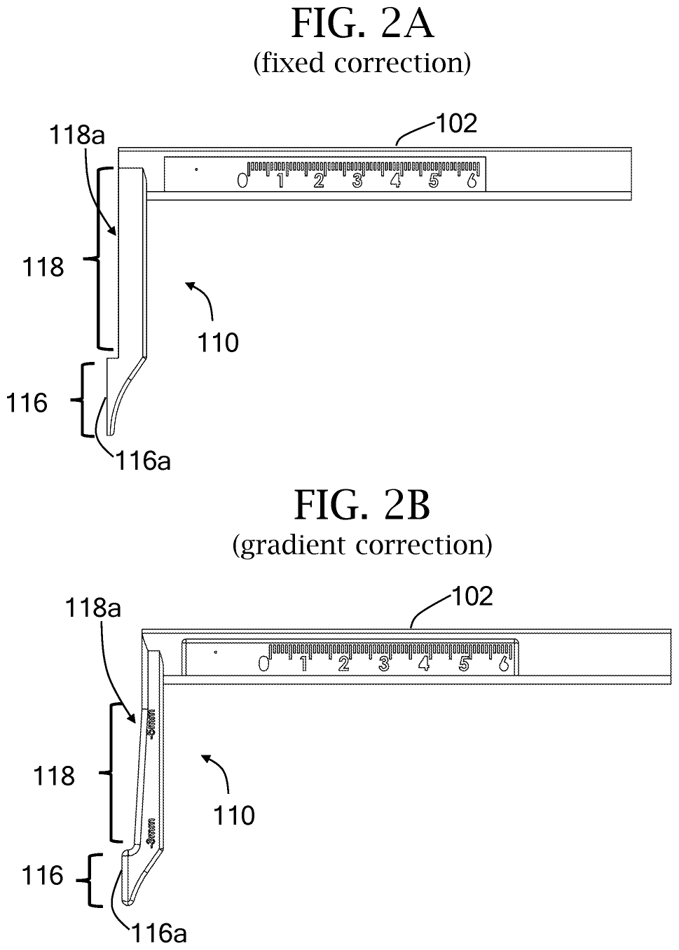

FIG. 2A shows a version of the first jaw 110, in greater detail, with a built-in “fixed correction”.

A distal portion 116 of the first jaw 110 has an outer (left, as viewed) surface 116a that is flat, or straight, which is substantially perpendicular to the longitudinal axis of the elongate member 102, and which may also be substantially parallel to the inner surface 120a (left, as viewed) of the second jaw 120.

A proximal portion 118 of the first jaw 110 has an outer (left, as viewed) surface 118a that is flat, or straight, which is substantially perpendicular to the longitudinal axis of the elongate member 102, and which may also be substantially parallel to the inner surface 120a (left, as viewed) of the second jaw 120.

The built-in “fixed correction” results from the proximal portion 118 of the first jaw 110 being offset, such as 3 mm inward (towards the second jaw 120) from the distal portion 116.

In use, the first measurement (Step 1. measure the eye-to-ear distance) may be performed by aligning the distal portion 116 of the first jaw 110 with the patient's eye, or ear. And, aligning the second jaw 120 with the patient's ear, or eye. Thereby determining the patient's eye-to-ear distance.

Knebelman appears to use the inner side of the fixed jaw in the nose. This could be accommodated.

In use, the second measurement (Step 2. measuring the nose-to-chin distance) may be performed by aligning the proximal portion 118 of the first jaw 110 with the patient's nose, or chin. And, aligning the second jaw with the patient's chin, or nose. Thereby determining the patient's nose-to-chin distance.

FIG. 2B shows a version of the first jaw 110, in greater detail, with a built-in “gradient correction”.

In most regards, this version (2B) is identical with the previous version (2A), with the difference (exception) being that the proximal portion 118 of the first jaw 110 is sloped. In another words, rather than having a fixed 3 mm (e.g.) offset, the outer (left, as viewed) surface of the first jaw 110 is tapered, or sloped, so that:

where the proximal portion 118 meets the distal portion 116, the offset is 3 mm (e.g.); and

where the proximal portion is nearer to the elongate member 102, the offset is 5 mm (e.g.).

In use, by disposing the proximal portion 118 appropriately with regard to the patient's nose or chin, the second measurement (Step 2. measuring the nose-to-chin distance) may be made without reference to an external table (e.g., Knebelman's “Table A”). The “factored distance” is built into the caliper itself.

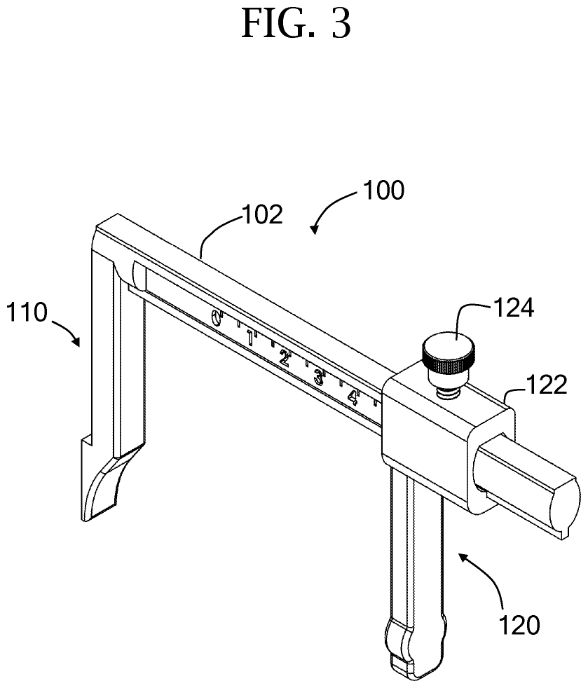

FIG. 3 shows the entire caliper, and the second jaw 120 in greater detail.

A sliding member (“slider”) 122 is disposed loosely (i.e., free to move) on or about the elongate member 102, and may be secured to the elongate member 102 with a thumbscrew (set screw) 124. A spring may be provided between the thumbscrew 124 and the beam 102 to tension the thumbscrew 124.

In this view, the elongate member 102 is shown having a somewhat oval cross-section (or profile). The profile could be a simple rectangle, or any other suitable cross-sectional shape.

### Some Final Comments

Knebelman discussed performing a “factoring” step between the two anatomical measurements (1. eye-to-ear; 2. nose-to-chin). This is no longer necessary, using the dental caliper and method disclosed herein.

Knebelman also disclosed having two scales, and referring to a table to establish an offset. This is no longer necessary, using the dental caliper and method disclosed herein.

An embodiment where the “fixed correction” or “gradient correction” is mounted to the movable member may also yield the same measurement results.

### APPENDIX

See http://craniometers.com/ for a presentation of how Knebelman works.

While the invention(s) has/have been described with respect to a limited number of embodiments, these should not be construed as limitations on the scope of the invention(s), but rather as examples of some of the embodiments. Those skilled in the art may envision other possible variations, modifications, and implementations that are also within the scope of the invention(s), and claims, based on the disclosure(s) set forth herein.

What is claimed is:

1. Dental caliper for determining occlusion, comprising:
an elongate member, or “beam”, marked with a scale (indicia);
a first jaw (or probe) extending from the beam, and fixed to the beam; and
a second jaw (or probe) extending from the beam, and movable along the beam;
wherein:
a proximal portion of the first (fixed) jaw is offset from a distal portion of the jaw.

2. The dental caliper of claim 1, wherein:
the offset is fixed, such as 3 mm.

3. The dental caliper of claim 1, wherein:
the offset has a gradient, such as 3-5 mm.

4. Method of determining vertical dimension of occlusion, comprising:
using the caliper of claim 1, performing the following steps:
Step 1. measuring the eye-to-ear distance (corresponds with Knebelman Step 1); and
Step 2. measuring the nose-to-chin distance (corresponds with Knebelman Step 3);
without adjusting the probes (Knebelman Step 2).
